(19) United States
(12) Patent Application Publication
(10) Pub. No.: US 2020/0299635 A1
(43) Pub. Date: Sep. 24, 2020

(54) ALGAE CULTIVATION MEDIUM AND METHOD OF INCREASING CARBON SHUTTLING IN AN ALGEA CULTIVATION MEDIUM

(71) Applicant: University of Kentucky Research Foundation, Lexington, KY (US)

(72) Inventors: Jesse Thompson, Lexington, KY (US); Leland Widger, Lexington, KY (US); Michael Wilson, Lexington, KY (US); Mark Crocker, Lexington, KY (US); Moushumi Sarma, Lexington, KY (US); Kunlei Liu, Lexington, KY (US)

(21) Appl. No.: 16/825,281

(22) Filed: Mar. 20, 2020

Related U.S. Application Data
(60) Provisional application No. 62/821,087, filed on Mar. 20, 2019.

(51) Int. Cl. C12N 1/12; C12N 5/00

(52) U.S. Cl. CPC C12N 1/12; C12N 2500/46; C12N 5/0025

(57) ABSTRACT

An algae cultivation medium includes a growth medium and at least one of an amine additive and a water-soluble biomimetic catalyst. A related method of increasing carbon shuttling in an algae cultivation medium includes adding at least one of the amine additive and the water-soluble biomimetic catalyst to the algae cultivation medium.

### RELATED APPLICATION

This application claims priority to U.S. Provisional Patent Application Ser. No. 62/821,087 filed on Mar. 20, 2019 which is hereby incorporated by reference in its entirety.

### TECHNICAL FIELD

This document relates generally to the cultivation of algae and, more particularly to a new and improved algae cultivation medium as well as to a new and improved method of increasing carbon shuttling in an algae cultivation medium.

### BACKGROUND

Use of microalgae for the capture and utilization of anthropogenic carbon dioxide (CO₂) emissions is a well-studied strategy that has been implemented at the pilot scale in several locations globally. Waste streams from point generation sources (e.g. fossil fuel power plants) are used as a feedstock to grow biomass that is processed to biofuels, chemicals and other products. However the kinetics of CO₂ uptake are slow, limited by the concentration of carbon available for algae uptake in solution. In order to improve CO₂ utilization in algae cultivation systems, the algae cultivation medium and method disclosed herein improves the rate of CO₂ uptake in the cultivation medium and improves the equilibrium concentration of dissolved CO₂. As a result, CO₂ availability to the cultivation is improved, ensuring that the concentration of dissolved CO₂ is not rate limiting and that algae productivity is maximized. Moreover, for sparged cultivation systems, the increased CO₂ solubility and transfer rate of CO₂ to solution facilitates improved CO₂ dissolution, and hence utilization, i.e., comparatively less CO₂ is lost to the gas phase.

### SUMMARY

In accordance with the purposes and benefits described herein, a new and improved algae cultivation medium is provided. That algae cultivation medium comprises a growth medium of a type providing a favorable environment for the growth of the algae in combination with (a) an amine additive, (b) a water soluble biomimetic catalyst or (c) an amine and a water soluble biomimetic catalyst.

The amine additive may be a hindered primary amine, a hindered secondary amine, a tertiary amine or an amino acid. The biomimetic catalyst may be selected from a group of compounds consisting of those listed in Table 1 and combinations thereof (see Table 1 below).

In accordance with yet another aspect, a new and improved method of increasing carbon shuttling in an algae cultivation medium comprises the step of adding (a) an amine additive, (b) a water soluble biomimetic catalyst or (c) an amine additive and a water soluble biomimetic catalyst to the algae cultivation medium.

The amine additive may be a hindered primary amine, a hindered secondary amine, a tertiary amine or an amino acid. The biomimetic catalyst may be selected from a group of compounds consisting of those listed in Table 1 and combinations thereof.

The amine additive may be added to reach a concentration in the algae cultivation medium of between about 0.05-1.0 percent by weight and more typically between about 0.1 and about 0.5 percent by weight. The water soluble biomimetic catalyst may be added to reach a concentration in the algae cultivation medium of between about 0.01 and about 0.5 percent by weight.

### DETAILED DESCRIPTION

The new and improved algae cultivation medium disclosed in this document comprises a growth medium in combination with (a) an amine additive, (b) a water soluble biomimetic catalyst or (c) an amine additive and a water soluble biomimetic catalyst.

The growth medium may comprise any growth medium of a type known in the art for growing and sustaining the algae of interest. Such a growth medium includes, but is not necessarily limited to, a urea-based cultivation medium, Bold's Basal Medium (BBM), Bristol medium, BG-11 medium, F/2 medium, Allens's blue-green medium and combinations thereof.

The algae of interest may include, but are not necessarily limited to, microalgae, encompassing Chlorophyta (green algae), Phaeophyta (brown algae), Pyrrophyta (dinoflagellates), Chrysophyta (diatoms), Rhodophyta (red algae), Euglenophyta (euglenoids), cyanobacteria (blue-green algae) and macroalgae (seaweed). Examples of commercially important algae include, but are not limited to, Chlorella sp., Scenedesmus sp., Nannochloropsis sp., Haematococcus sp., Dunaliella sp., Crypthecodinium sp., Schizochytrium sp., Phaeodactylum sp., Nitzschia sp. and Porphyridium sp. Examples of commercially important cyanobacteria include Spirulina sp.

It is believed that carbon dioxide (CO₂) is the preferred species for carbon uptake in algal species. However, in aqueous solution at biological pH, bicarbonate (HCO₃⁻) is the dominant species and relatively small concentrations of dissolved CO₂ are available for CO₂ uptake, based upon the equilibrium between HCO3⁻ and CO₂. Significantly, increasing the concentration of bicarbonate in solution will, by LeChatlier's principle, also increase the dissolved CO₂ concentration due to the fixed equilibrium constant. Advantageously, the amine additive effectively increases the soluble concentration of bicarbonate, and thereby the dissolved CO₂, in the algae cultivation medium.

In one or more of the many possible embodiments of the algae cultivation medium, the amine additive is a hindered primary amine. Hindered primary amines useful for this purpose include, but are not necessarily limited to, 2-Amino-2-Methyl-Propanol (AMP), 1-Amino-2-Propanol (1A2P), 2-Amino-1-Propanol (2A1P) and combinations thereof.

In one or more of the many possible embodiments of the algae cultivation medium, the amine additive is a hindered secondary amine. Hindered secondary amines useful for this purpose include, but are not necessarily limited to, N-Methylethanolamine (NMEA), 2-(Ethylamino) ethanol (EAE), Diethanolamine (DEA), 3-(Methylamino) propylamine (MAPA) and combinations thereof.

In one or more of the many possible embodiments of the algae cultivation medium, the amine additive is a tertiary amine. Tertiary amines useful for this purpose include, but are not necessarily limited to, Triethanolamine (TEA), N-Methyldiethanolamine (MDEA), Dimethylethanolamine (DMEA) and combinations thereof.

In one or more of the many possible embodiments of the algae cultivation medium, the amine additive is an amino acid. Amino acids useful for this purpose include, but are not necessarily limited to, N,N-dimethylglycine (DMG), β-Alanine, Alanine, Glycine, Sarcosine, Taurine, L-Serine, L-Proline and combinations thereof.

The water soluble biomimetic catalyst may be selected from a group of compounds consisting of those listed in Table 1 and combinations thereof (see below).

TABLE 1
List of Catalysts

| | | Water | |
|---|---|---|---|
| Catalyst | | Soluble | |
| Name | Metal | Group | Structure |
| C1P | Co | [PPh₃]Cl | 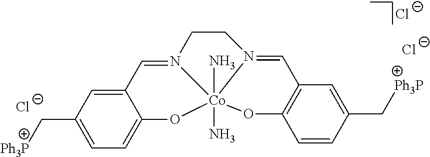 |
| C1P* | Co | [P(OEt)₃]Cl | 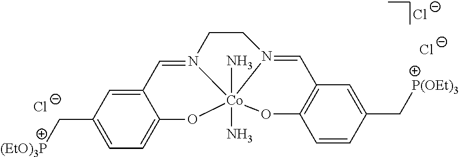 |
| C1I | Co | [Imidazol]PF₆ | 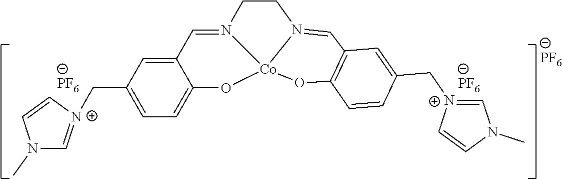 |
| C1I* | Co | [Imidazol]Cl | 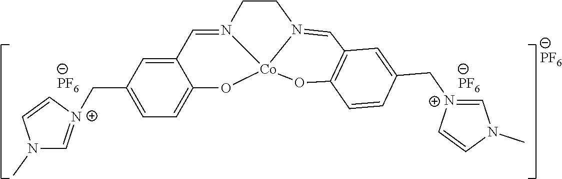 |
| C2 | Zn | none | 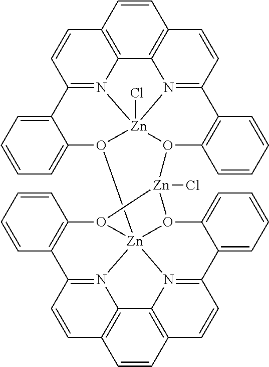 |
| C3I | Zn | [Imidazol]PF₆ | 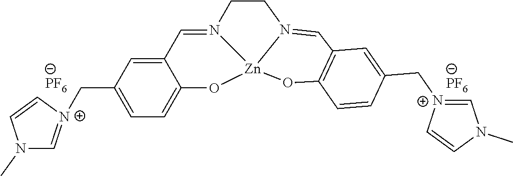 |
| C3I* | Zn | [Imidazol]Cl | 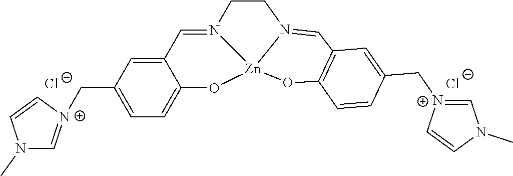 |
| C3P | Zn | [PPh₃]Cl | 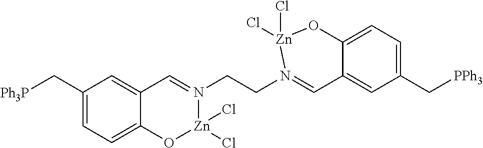 |
| C3P* | Zn | [P(OEt)₃]Cl | 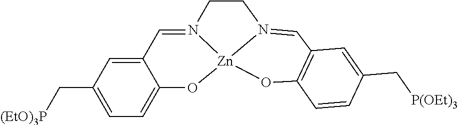 |
| C3Pr | Zn | [PPh₃]Cl | 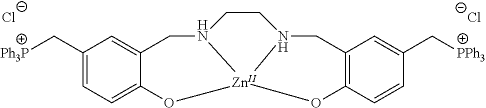 |
| C4 | Zn | COOH | 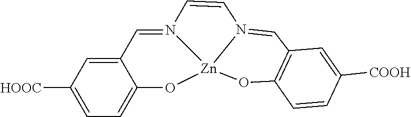 |
| C5z | Zn | COOH | 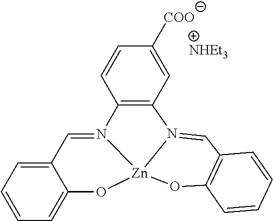 |
| C5zr | Zn | COOH | 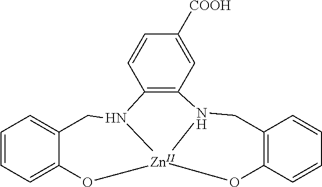 |
| C5c | Co | COOH | 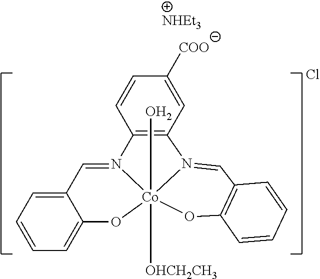 |
| C6z | Zn | COOH | 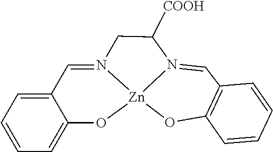 |
| C7z | Zn | [PPh3]Cl | 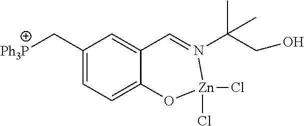 |
| C7z(BF4) | Zn | [PPh3]BF₄ | 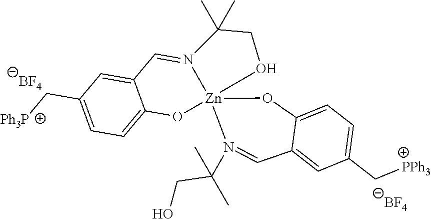 |
| C7c | Co | [PPh3]Cl | 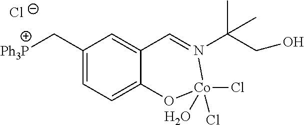 |
| C8z | Zn | [PPh3]Cl | 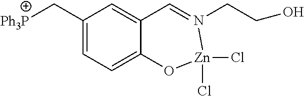 |
| C8c | Co | [PPh3]Cl | 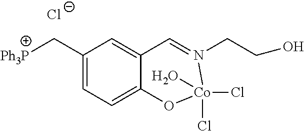 |
| C9z | Zn | | 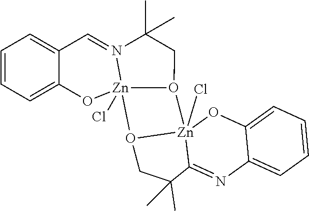 |
| C10z | Zn | | 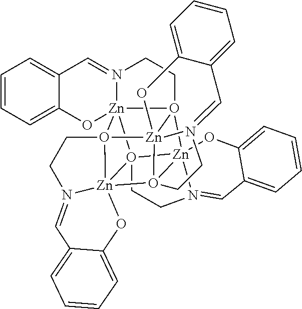 |
| C11z | Zn | OH | 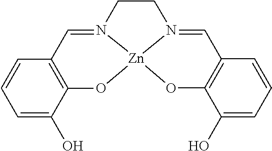 |
| C11c | Co | OH | 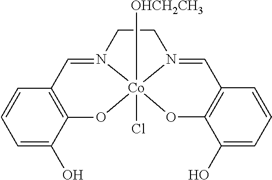 |
| C11n | Ni | OH | 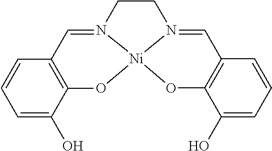 |
| C12z | Zn | | 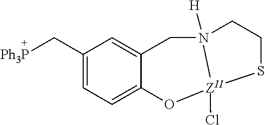 |
| C12c | Co | | 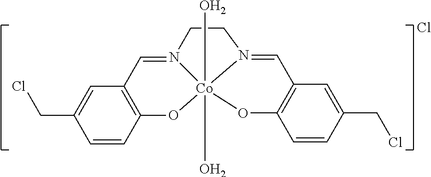 |
| C13z | Zn | OH | 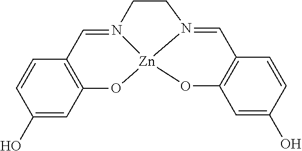 |
| C13c | Co | OH | 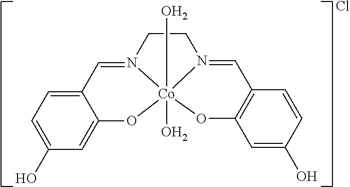 |
| C14z | Zn | OH and COOH | 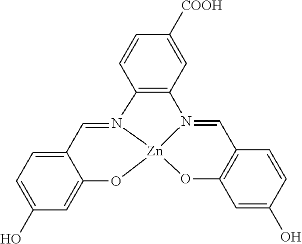 |
| C14c | Co | OH and COOH | 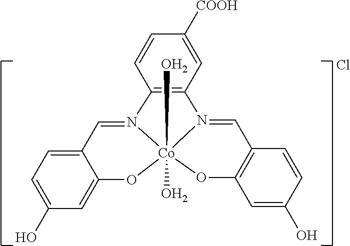 |
| C15z | Zn | OH | 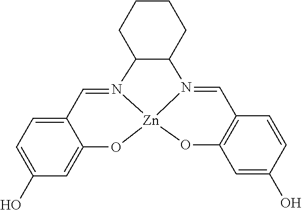 |
| C15c | Co | OH | 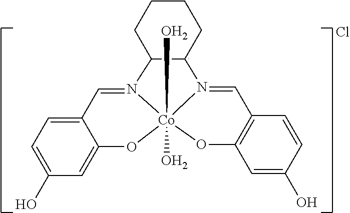 |

Details and the synthesis of many of these catalysts were previously described in U.S. Pat. No. 10,213,734, the full disclosure of which is incorporated herein by reference.

Such water soluble biomimetic catalysts may incorporate water solubilizing groups on the ligand backbone for use in aqueous growth media and exhibit desired complex stability, solubility in aqueous amine solvents and electronic properties of the catalytically active metal center. As a result, the biomimetic catalysts increase the rate of algal CO₂ uptake by shuttling CO₂ (as bicarbonate) into solution, increasing the local concentration and ensuring CO₂ concentration is not rate limiting. More specifically, these catalysts incorporate key functional groups that facilitate CO₂ binding, enhance CO₂ hydration, encourage bicarbonate dissociation and increase water solubility.

In one or more of the many possible embodiments of the algae growth medium, the amine additive is provided at a concentration of between about 0.05 to about 1.0 percent by weight and more typically between about 0.1 and about 0.5 percent by weight.

In one or more of the many possible embodiments of the algae growth medium, the water soluble biomimetic catalyst is provided at a concentration of between about 0.01 and about 0.5 percent by weight.

When culturing algae with added water soluble biometric catalyst but no amine additive, the cultivation medium pH should optimally be maintained at a value such that CO₂ solubility is maximized, but is not so high as to result in metabolic inhibition of the organism. In practice, optimal pH value will typically lie in the range of pH 9-11. Such conditions can be achieved by the addition of base to the cultivation, such as Na₂CO₃, NaHCO₃, K₂CO₃, KHCO₃, etc.

In one or more of the many possible embodiments of the algae cultivation medium, the amine additive is provided at a concentration of between about 0.05 to about 1.0 percent by weight and more typically between about 0.1 and about 0.5 percent by weight and the water soluble biomimetic catalyst is provided at a concentration of between about 0.01 and about 0.5 percent by weight. Useful weight ratios for the amount of amine additive added to the amount of water soluble biometric catalyst added when used together may range from about 2:1 to about 20:1.

Consistent with the above description, a method for increasing carbon shuttling in an algae cultivation medium includes the step of adding (a) an amine additive, (b) a biomimetic catalyst or (c) an amine additive and a biomimetic catalyst to the algae cultivation medium.

The method may further include the step of using a hindered primary amine, a hindered secondary amine, a tertiary amine, an amino acid or a combination thereof as the amine additive. Representative hindered primary amines, hindered secondary amines, tertiary amines and amino acids are identified above.

The method may further include the step of using as the water soluble biometric catalyst one or more of the particular compounds identified above in Table 1.

In one or more particularly useful embodiments, the method includes the addition of both (a) the amine additive, in the form of at least one hindered primary amine, at least one hindered secondary amine, at least one tertiary amine, at least one amino acid or a combination thereof, and (b) the biomimetic catalyst.

In any of the many possible embodiments, the amine additive may be added to the algae cultivation medium at a concentration of between about 0.05 to about 1.0 percent by weight and more typically between about 0.1 and about 0.5 percent by weight. In any of the many possible embodiments, the water soluble biomimetic catalyst may be added to the algae cultivation medium at a concentration of between about 0.01 and about 0.5 percent by weight. In many possible embodiments, the amine additive and the water soluble biometric catalyst are both added to the algae cultivation medium at the indicated concentrations.

The new and improved algae cultivation medium and method of increasing carbon shuttling in an algae cultivation medium would benefit commercial photosynthetic algae and cyanobacteria producers seeking to improve their carbon dioxide utilization efficiencies. Sources for carbon dioxide include, but are not limited to, (a) industrial CO₂ point sources, including flue gas from coal-, oil- and natural gas-fired power plants, boilers, furnaces, cement kilns, chemical plants, steel plants, bioethanol plants and the like, (b) concentrated CO₂ streams obtained from CO₂ concentration processes and (c) air containing CO₂ at atmospheric concentration.

Principle benefits include but are not necessarily limited to low capital cost, together with the relatively simple operation to increase carbon availability and CO₂ absorption kinetics/utilization efficiency. The application of the technology would not require any modification to the cultivation infrastructure (ponds or PBRs) or acquisition of specialized equipment. This represents a significant advantage over approaches that incorporate the use of CO₂ scrubbers or other CO₂ concentrating technologies.

The foregoing has been presented for purposes of illustration and description. It is not intended to be exhaustive or to limit the embodiments to the precise form disclosed. Obvious modifications and variations are possible in light of the above teachings. All such modifications and variations are within the scope of the appended claims when interpreted in accordance with the breadth to which they are fairly, legally and equitably entitled.

What is claimed:

1. An algae cultivation medium comprising:
a growth medium; and
an amine additive, (b) a water soluble biomimetic catalyst or (c) an amine additive and a water soluble biomimetic catalyst.

2. The algae cultivation medium of claim 1, wherein said amine additive is a hindered primary amine.

3. The algae cultivation medium of claim 1, wherein said amine additive is a hindered secondary amine.

4. The algae cultivation medium of claim 1, wherein said amine additive is a tertiary amine.

5. The algae cultivation medium of claim 1, wherein said amine additive is an amino acid.

6. The algae cultivation medium of claim 1, wherein said biomimetic catalyst is selected from a group of catalysts consisting of

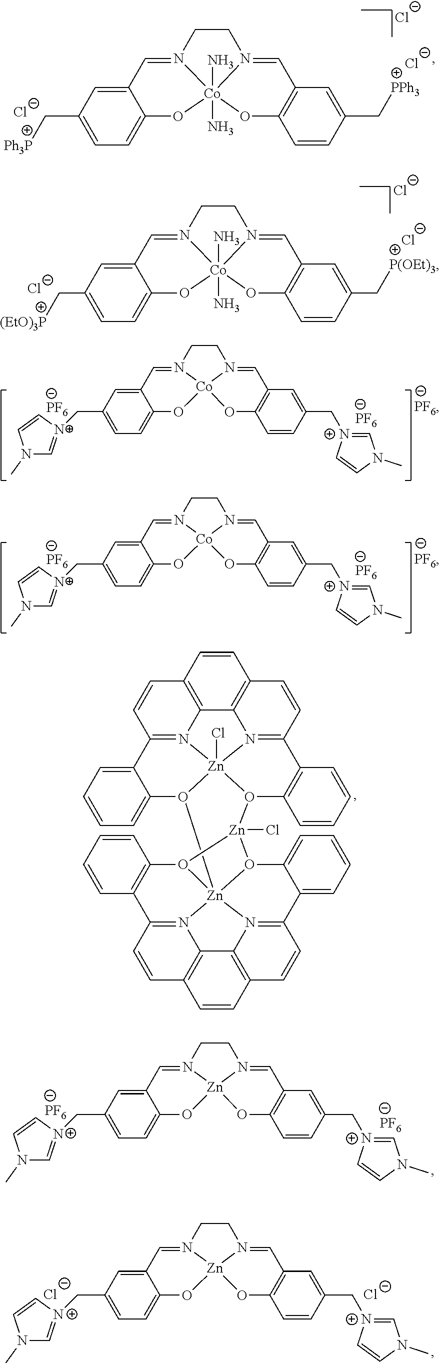

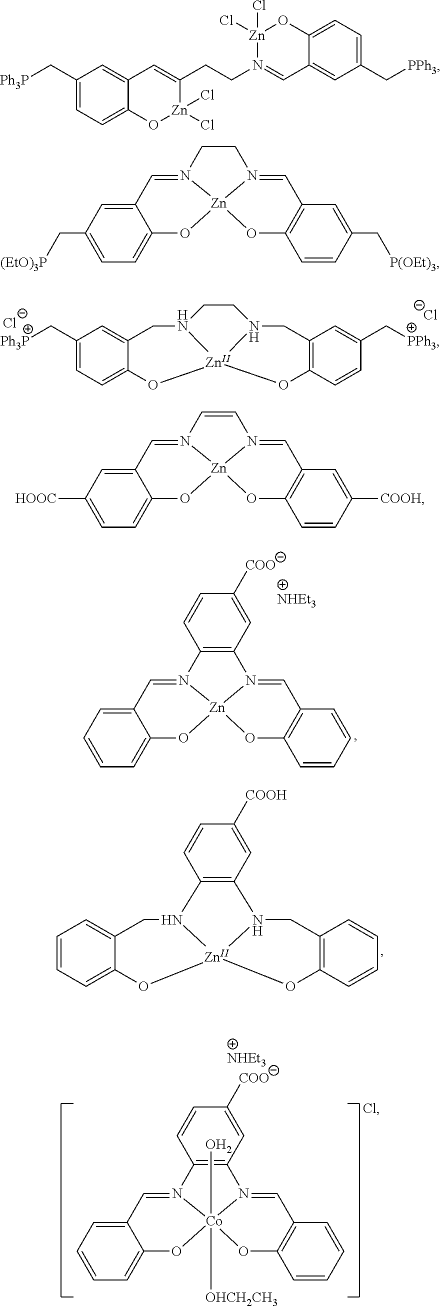

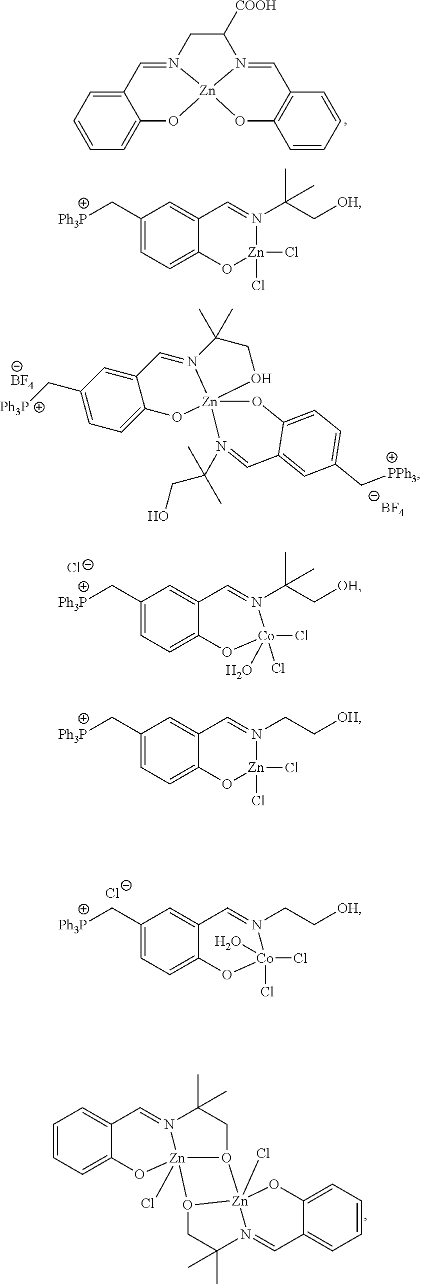

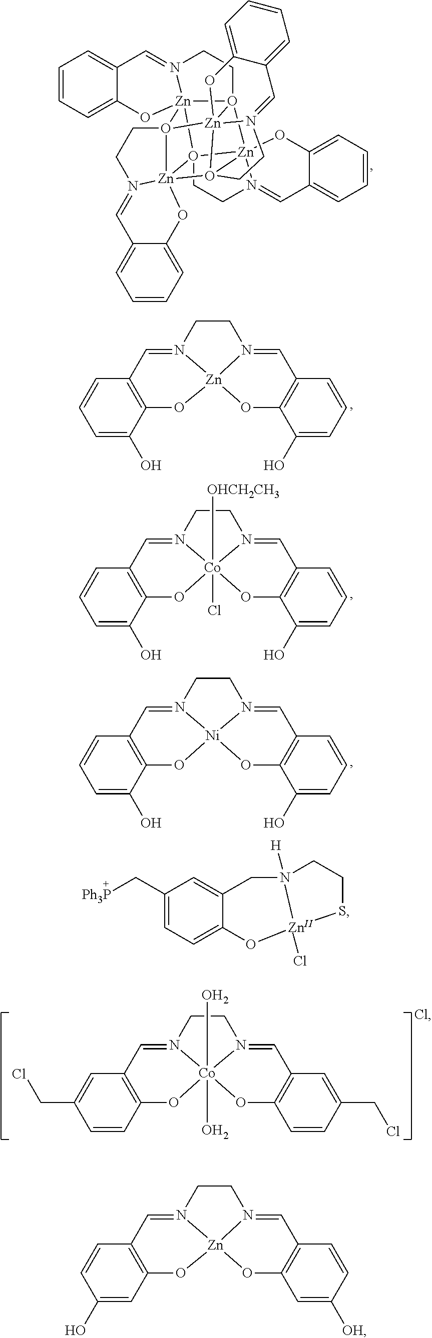

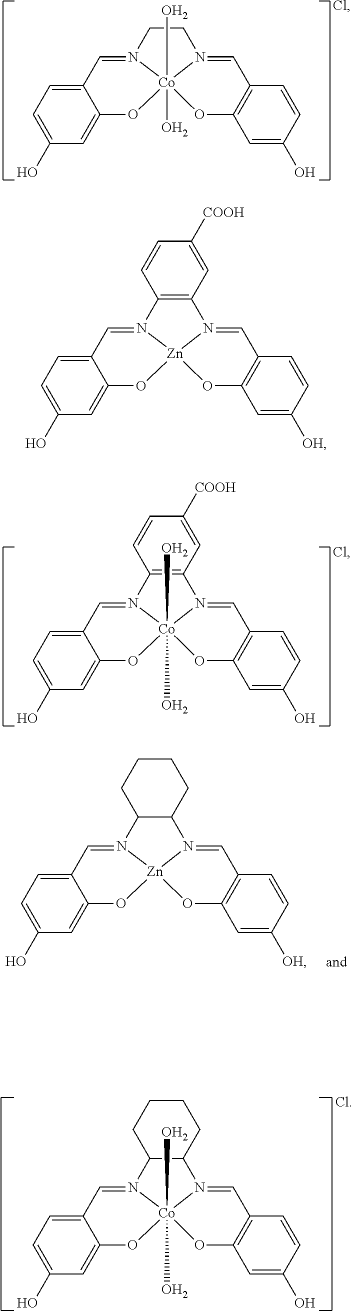

7. The algae cultivation medium of claim 1 including (a) an amine additive selected from a group consisting of a hindered primary amine, a hindered secondary amine, a tertiary amine, an amino acid and combinations thereof and (b) a water soluble biomimetic catalyst.

8. The algae cultivation medium of claim 7, wherein the biomimetic catalyst is selected from a group consisting of

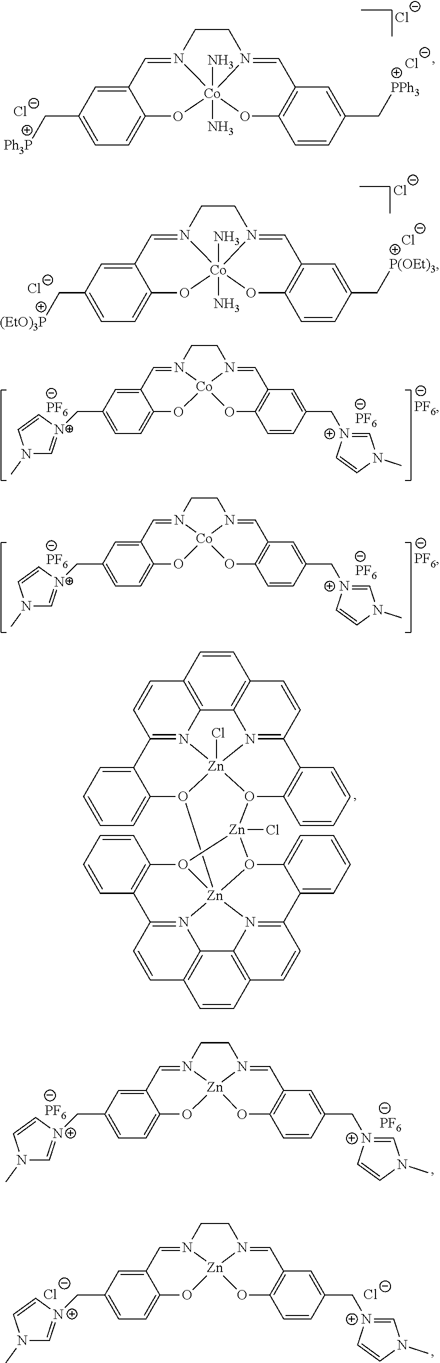

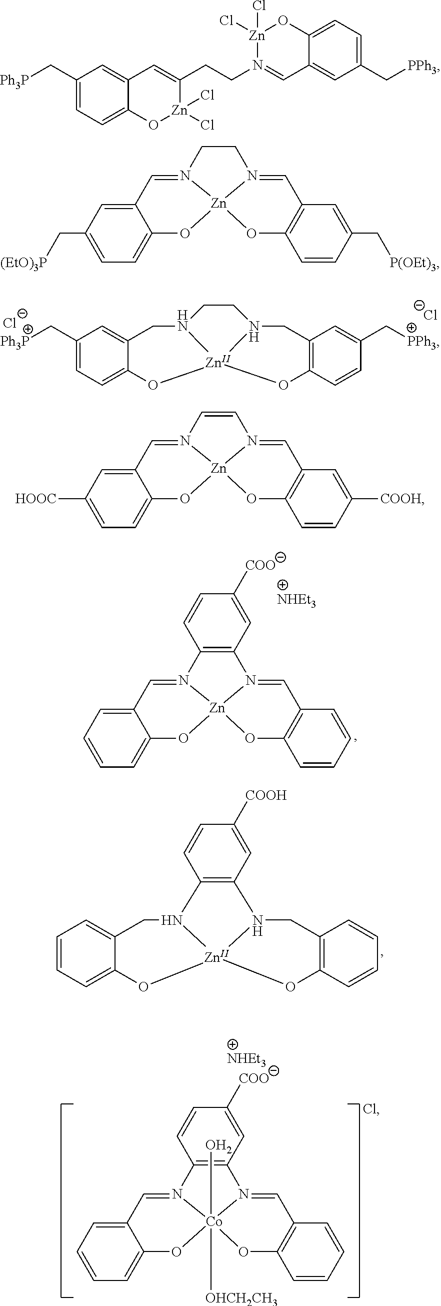

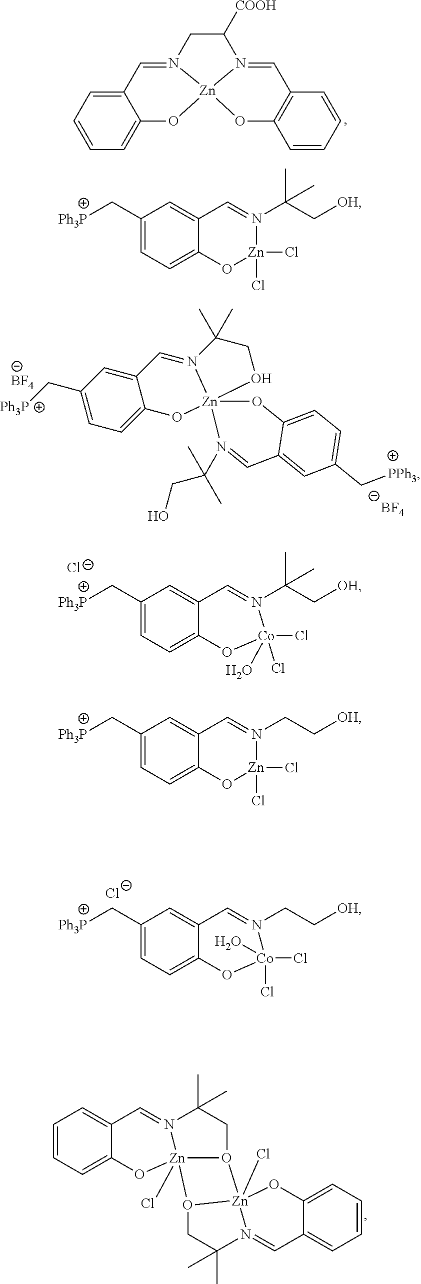

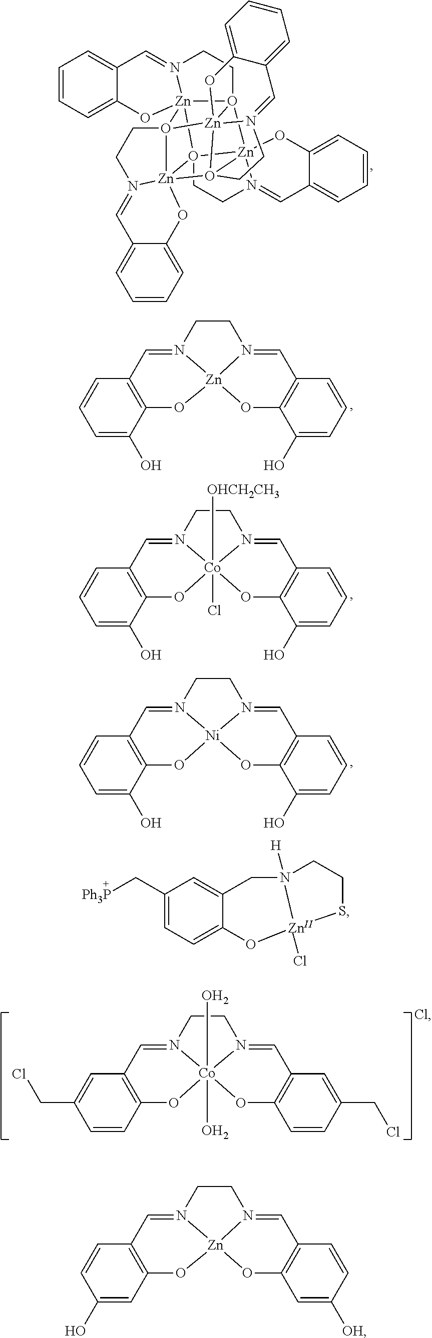

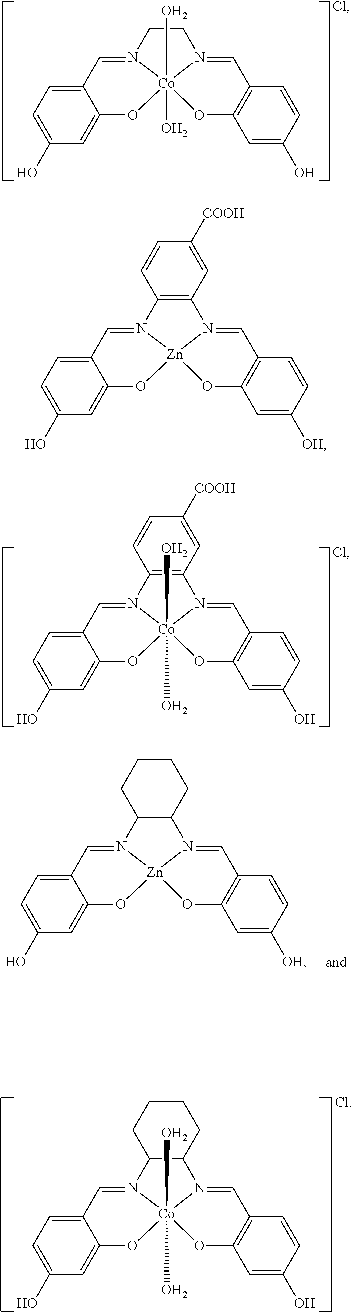

9. A method of increasing carbon shuttling in an algae cultivation medium, comprising:
adding (a) an amine additive, (b) a biomimetic catalyst or (c) an amine additive and a biomimetic catalyst to the algae cultivation medium.

10. The method of claim 9, including using a hindered primary amine as the amine additive.

11. The method of claim 9, including using a hindered secondary amine as the amine additive.

12. The method of claim 9, including using a tertiary amine as the amine additive.

13. The method of claim 9, including using an amino acid as the amine additive.

14. The method of claim 9, including using as the biomimetic catalyst a compound selected from a group consisting of 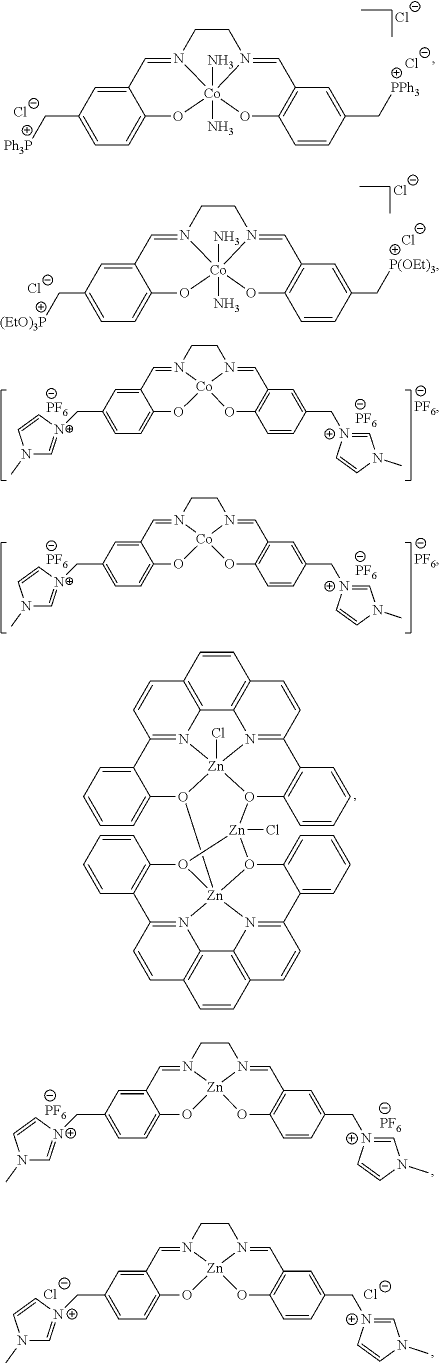 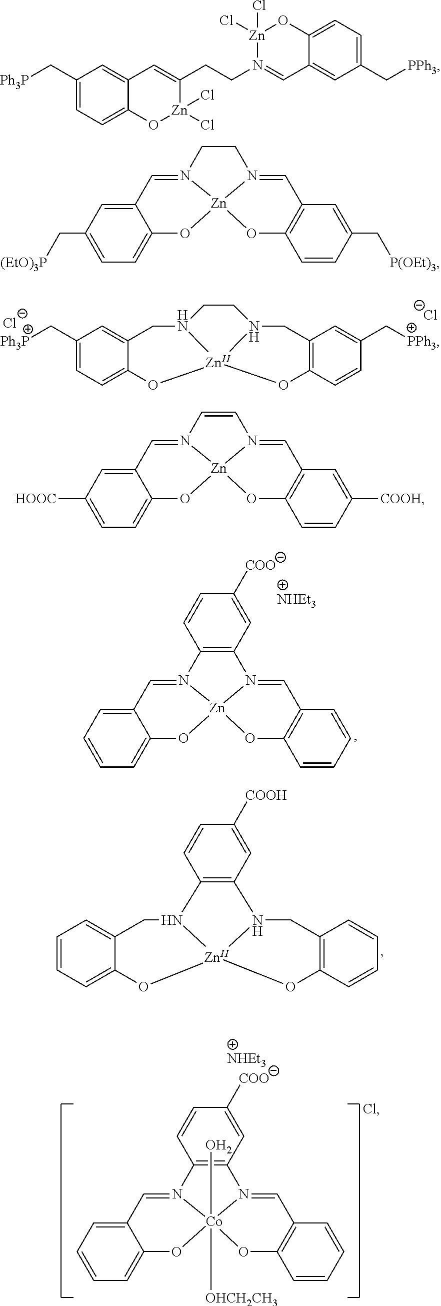 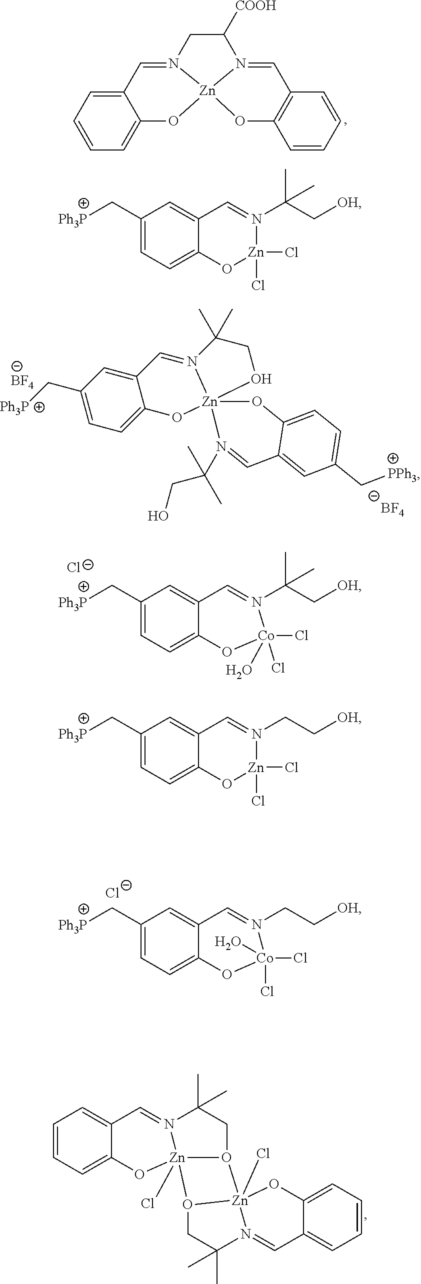 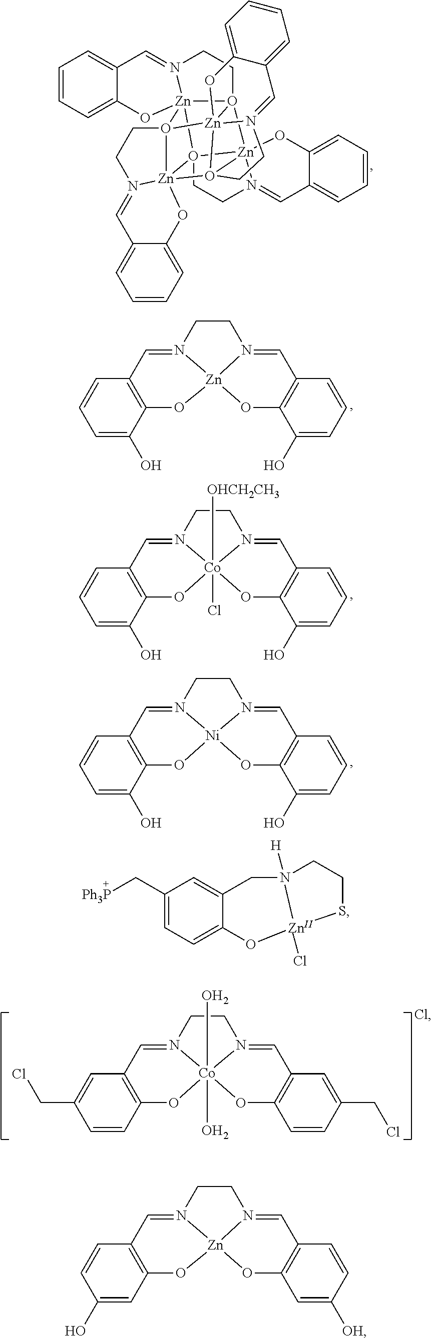 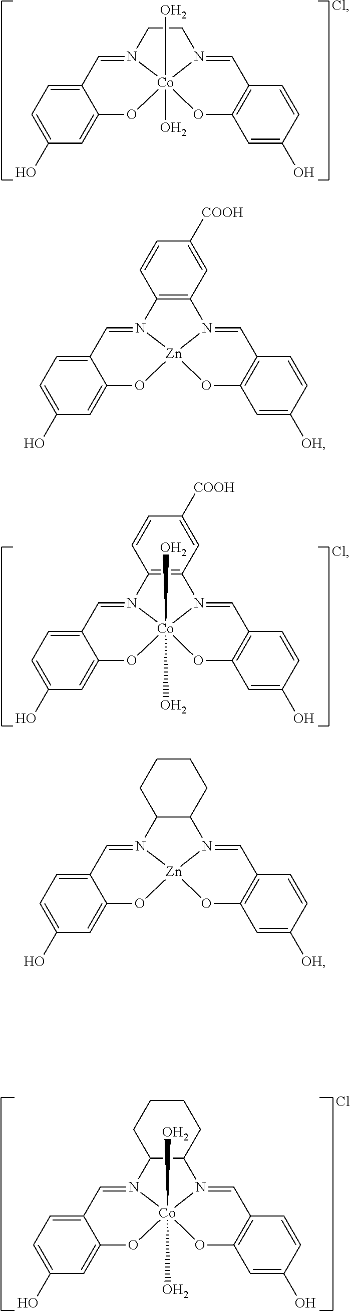
and combinations thereof.

15. The method of claim 9, including using a compound selected from a group consisting of a hindered primary amine, a hindered secondary amine, a tertiary amine, an amino acid and combinations thereof as the amine additive in conjunction with the biomimetic catalyst.

16. The method of claim 15, including using as the biomimetic catalyst a compound selected from a group consisting of 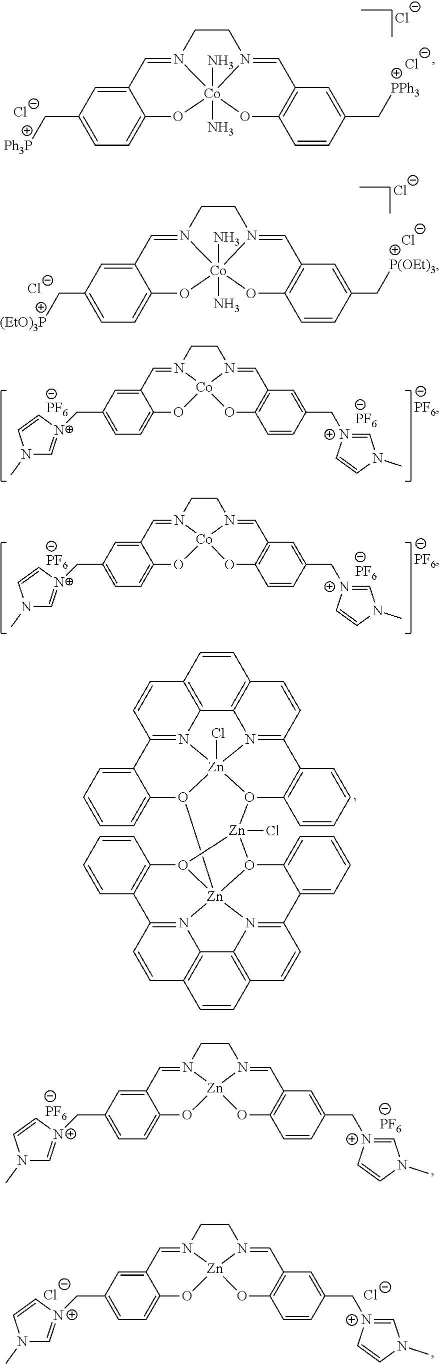 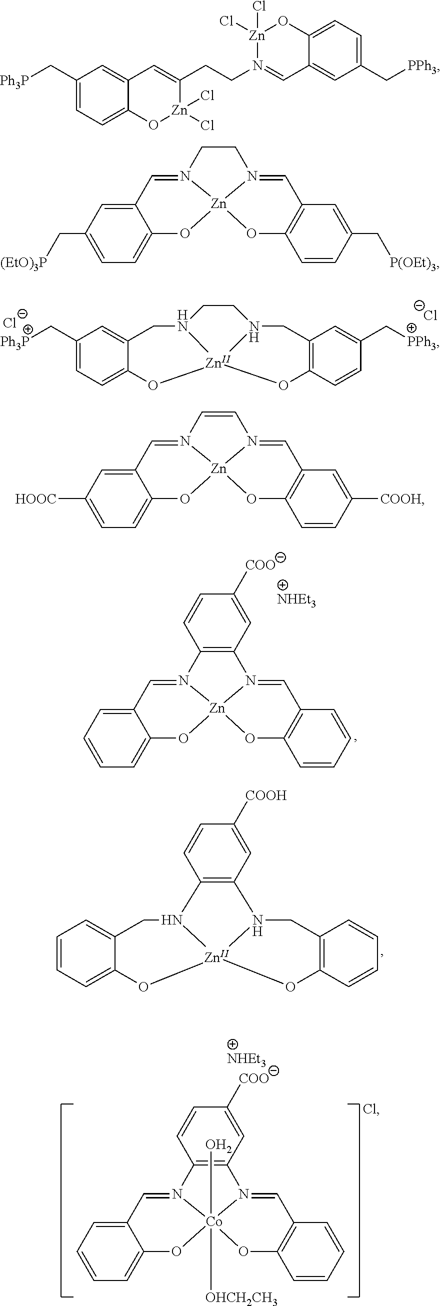 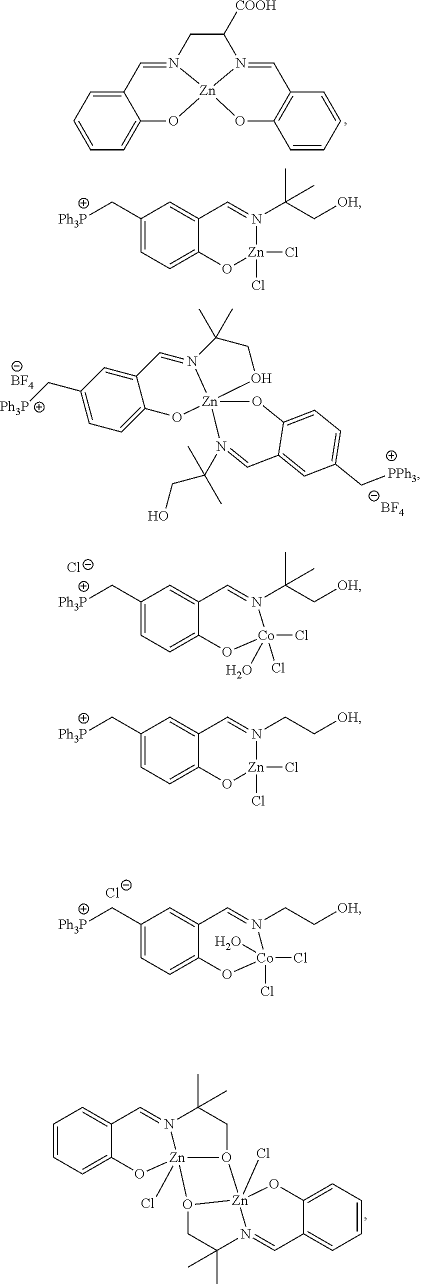 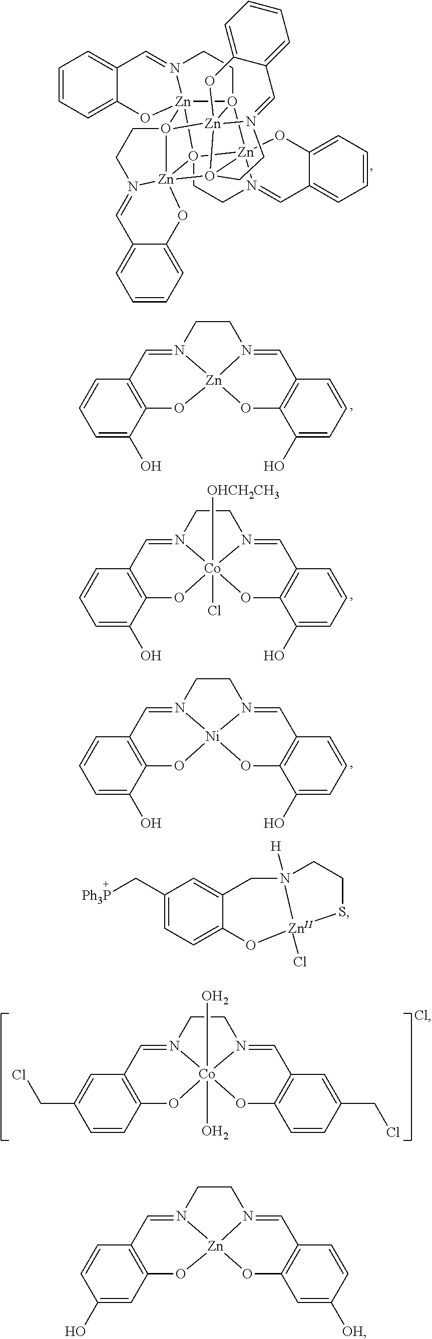 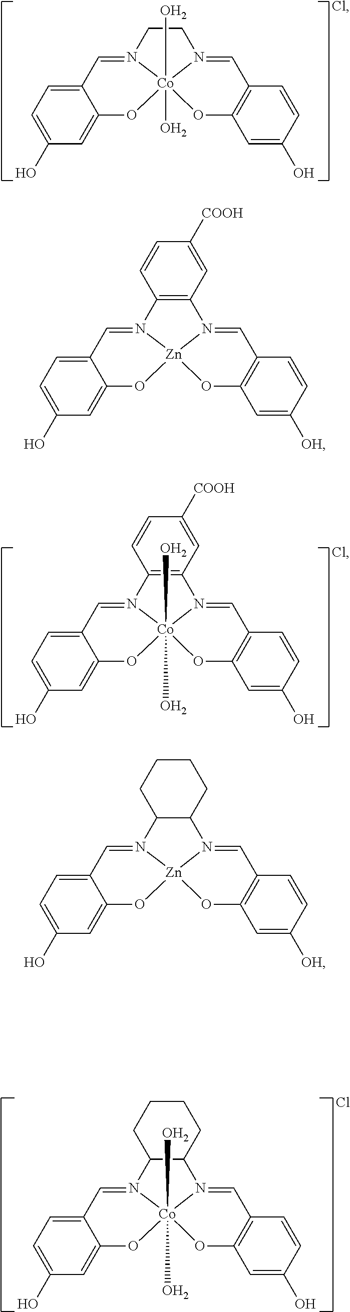
and combinations thereof.

17. The method of claim 16 including adding said amine to a concentration of between 0.05 and 1.0 percent by weight and adding said biomimetic catalyst to a concentration of between 0.01 and 0.5 percent by weight.

18. The method of claim 9 including adding said amine additive to a concentration of between 0.05 and 1.0 percent by weight.

19. The method of claim 9 including adding said biomimetic catalyst to a concentration of between 0.01 and 0.5 percent by weight.
